(19) United States
(12) Patent Application Publication
(10) Pub. No.: US 2011/0201927 A1
(43) Pub. Date: Aug. 18, 2011

(54) ULTRASONIC DIAGNOSTIC APPARATUS

(76) Inventors: Kazuhiko Hayakawa, Tokyo (JP); Akimasa Asai, Tokyo (JP); Koji Yanagihara, Tokyo (JP)

(21) Appl. No.: 13/028,977

(22) Filed: Feb. 16, 2011

(30) Foreign Application Priority Data
Feb. 17, 2010 (JP) 2010-032778

(51) Int. Cl. A61B 8/00

(52) U.S. Cl. 600/437

(57) ABSTRACT

An ultrasonic diagnostic apparatus includes a housing and an arm extending in a direction of front and back of the apparatus above the housing. The arm configured to rotate upwardly and downwardly and attaching to an attachment provided above the housing. The apparatus further includes an operation panel provided at a distal end of the arm. The attachment is oriented at a certain position from an upper surface of the housing such that the arm is rotatable upward and downward from a horizontal position.

### CROSS REFERENCE TO RELATED APPLICATIONS

This application claims the benefit of Japan Patent Application No. 2010-32778 filed Feb. 17, 2010, which is hereby incorporated by reference in its entirety.

### BACKGROUND OF THE INVENTION

The present invention relates to an ultrasonic diagnostic apparatus that can change height positions of an operation panel.

An ultrasonic diagnostic apparatus includes an ultrasonic probe for acquiring echo signals by transmitting ultrasonic waves to a subject, a housing enclosing a circuit board including an arithmetic processor to create an ultrasonic image data based on the echo signals acquired by the ultrasonic probe, a display for showing the ultrasonic image based on the ultrasonic image data, and an operation panel for inputting commands by an operator.

The ultrasonic diagnostic apparatus including the above-mentioned configuration in which an operation panel is rotatable upward or downward to change its height is disclosed in Japanese Patent No. 4393506. A pair of arm members is attached to the operation panel of the ultrasonic diagnostic apparatus of Japanese Patent No. 4393506 and the operation panel can be moved upwardly and downwardly by rotating the arm members.

However, with the ultrasonic diagnostic apparatus disclosed in the US publication 20100094130A1, the length (in a direction of front and back) of entire apparatus becomes longer when the operation panel is moved downward than moved to upward. Then an installation space is needed.

### BRIEF DESCRIPTION OF THE INVENTION

An invention of first aspect is an ultrasonic diagnostic apparatus including a housing, an arm attached to an attachment provided above the housing so as to be rotatable upward and downward and extending in a direction of front and back of the apparatus located above the housing, an operation panel provided at a distal end of the arm. The attachment is provided at a certain position from an upper surface of the housing such that the arm is rotatable upward and downward from a position where the arm is in horizontal.

An invention of second aspect is that the ultrasonic diagnostic apparatus of the first aspect, wherein the attachment is provided on an arm attachment member protruded upward than an upper surface of the housing.

An invention of third aspect is that he ultrasonic diagnostic apparatus of the second aspect, wherein the arm attachment member is provided on a back side of the ultrasonic diagnostic apparatus and the arm is formed so as to extend from the back side to a front side.

An invention of forth aspect is that the ultrasonic diagnostic apparatus according to any of foregoing aspects, wherein a height of the attachment from the upper surface of the housing is defined such that a space for ensuring the rotating movement of the arm created above the housing.

An invention of fifth aspect is that the ultrasonic diagnostic apparatus according to any of foregoing aspects, wherein a rotating angle upward from a horizontal position and a rotating angle downward from the horizontal position of the arm are the same.

An invention of sixth aspect is that the ultrasonic diagnostic apparatus according to any of foregoing aspects, wherein rotating angle upward from a horizontal position and rotating angle downward from the horizontal position of the arm are different.

An invention of seventh aspect is that the ultrasonic diagnostic apparatus according to any of foregoing aspects, wherein wheels supporting the ultrasonic diagnostic apparatus at an installation surface are attached to wheel attachment members protruded laterally of the housing.

An invention of seventh aspect is that he ultrasonic diagnostic apparatus of the seventh aspect, wherein a height of attachment surface of the wheel on the wheel attachment member is higher than a lower surface of the housing.

An invention of ninth aspect is that the ultrasonic diagnostic apparatus according to any of foregoing aspects, wherein the housing is nearly cubical-shaped and has four side surfaces, and at least one connector for an ultrasonic probe is formed either on a right side surface or a left side surface adjacent to a front side surface, where an operator operates the operation panel.

An invention of tenth aspect is that the ultrasonic diagnostic apparatus according to any of foregoing aspects, wherein a display is attached to the operation panel via a display attaching arm, and the display attaching arm is attached to upper surface side of the operation panel and is formed upward so as not to interfere with the arm even when the arm rotates to the lowest position and the display attaching arm extends upper than the operation panel.

An invention of eleventh aspect is that the ultrasonic diagnostic apparatus according to any of foregoing aspects, wherein the arm is rotated by a parallel linking feature.

An invention of twelfth aspect is that the ultrasonic diagnostic apparatus according to any of foregoing aspects, wherein the housing encloses a circuit board including an arithmetic processor processing echo signals acquired by transmitting ultrasonic waves.

According to the above-mentioned inventions, the operation panel provided at the distal end of the arm can be rotated upwardly and downwardly by rotating the arm. Then, the arm is attached to the attachment provided at a higher position than the housing and is extended in the front-back direction of the apparatus, and also is rotatable upward and downward above the housing so that the length (a length in a direction of front and back) can be suppressed. Further, as the arm can rotate upwardly and downwardly from the horizontal position, the position difference in horizontal front and back direction of the operation panel by rotating the arm becomes smaller. Thus, the length (the length in the direction of the front and back) of the ultrasonic diagnostic apparatus is suppressed. And the ultrasonic diagnostic apparatus can be installed at a small space.

### BRIEF DESCRIPTION OF THE DRAWINGS

FIG. 1 is a perspective view showing an embodiment of an ultrasonic diagnostic apparatus of the present embodiment.

FIG. 2 is a front view of the ultrasonic diagnostic apparatus of FIG. 1.

FIG. 3 is a right side view of the ultrasonic diagnostic apparatus of FIG. 1.

FIG. 4 is a flat view of the ultrasonic diagnostic apparatus of FIG. 1.

FIG. 5 is a back-perspective view of the ultrasonic diagnostic apparatus of FIG. 1.

FIG. 6 shows an explanation of rotating angle of the arm of the ultrasonic diagnostic apparatus of FIG. 1.

FIG. 7A is a drawing illustrating that the arm is in the horizontal position.

FIG. 7B is a drawing illustrating that the arm rotated to the highest position.

FIG. 7C is a drawing illustrating that the arm rotated to the lowest position.

FIG. 8 is a right side view of the ultrasonic diagnostic apparatus where the arm rotates to the highest position.

FIG. 9 is a right side view of the ultrasonic diagnostic apparatus where the arm rotates to the lowest position.

FIG. 10A is a front view where an operator is in a squatting position to conduct scanning.

FIG. 10B is a flat view where an operator is in a squatting position to conduct scanning.

FIG. 11 is a perspective view of the ultrasonic diagnostic apparatus where the control panel is rotated horizontally from the position shown in FIG. 1.

FIG. 12 is a flat view of the ultrasonic diagnostic apparatus where the wheel attachment members are placed so as to be under a bed.

### DETAILED DESCRIPTION OF THE INVENTION

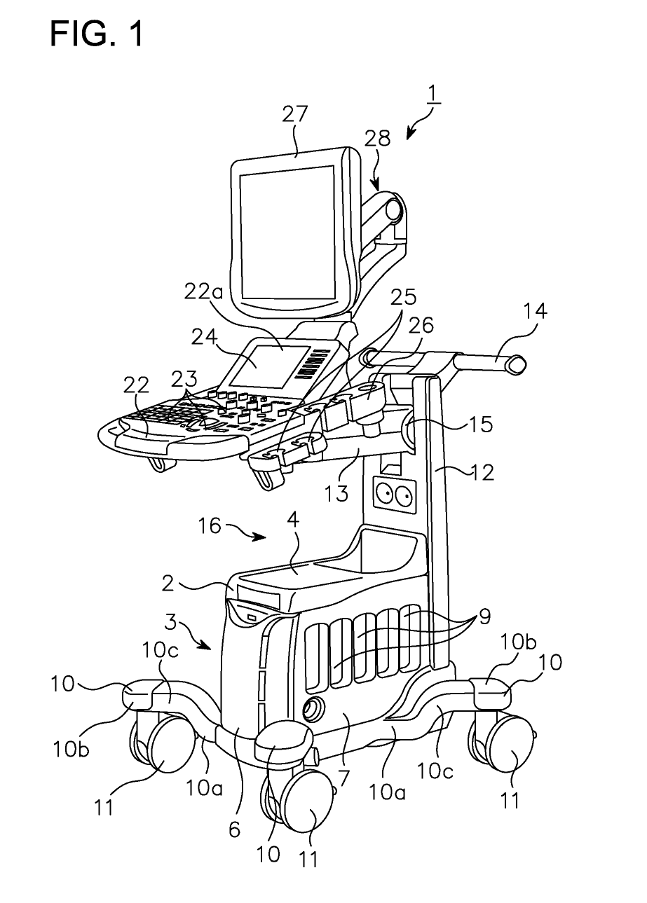

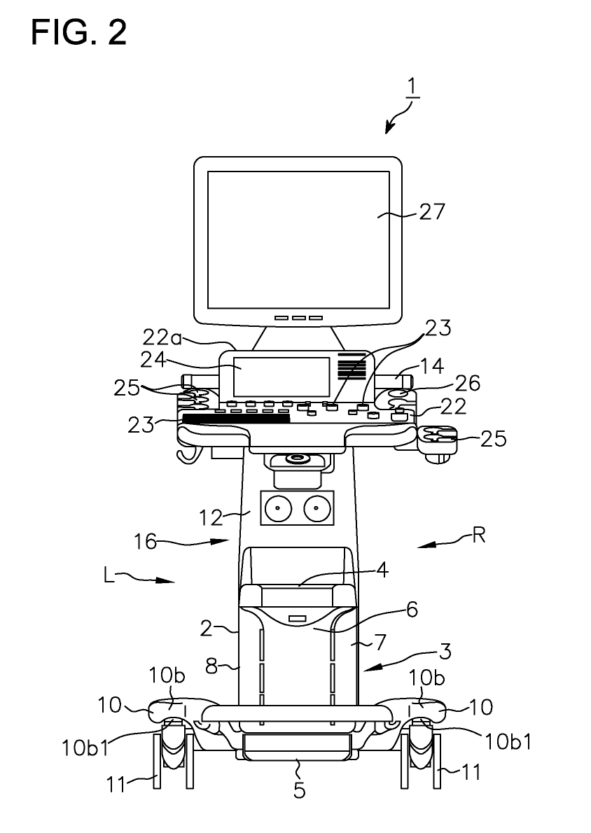

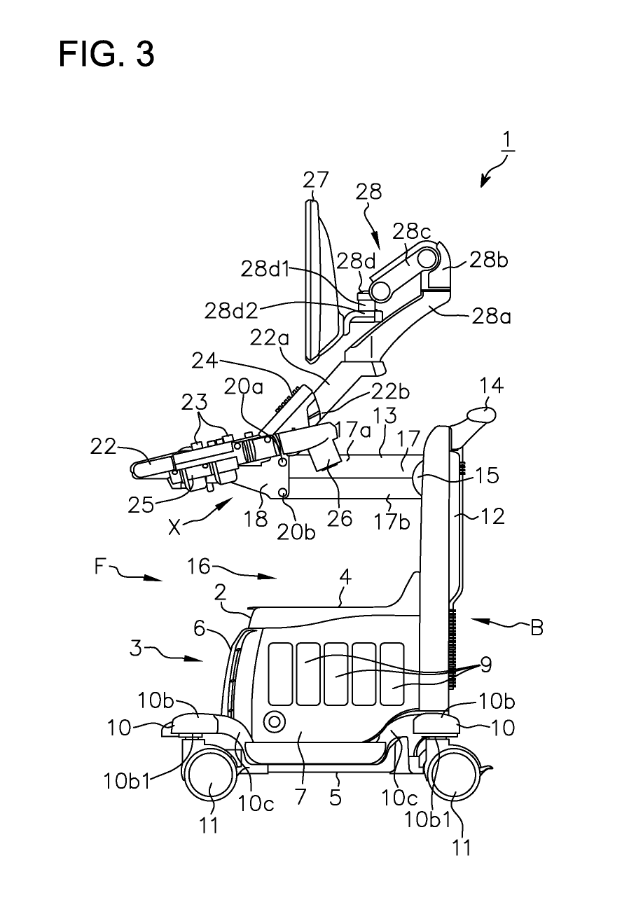

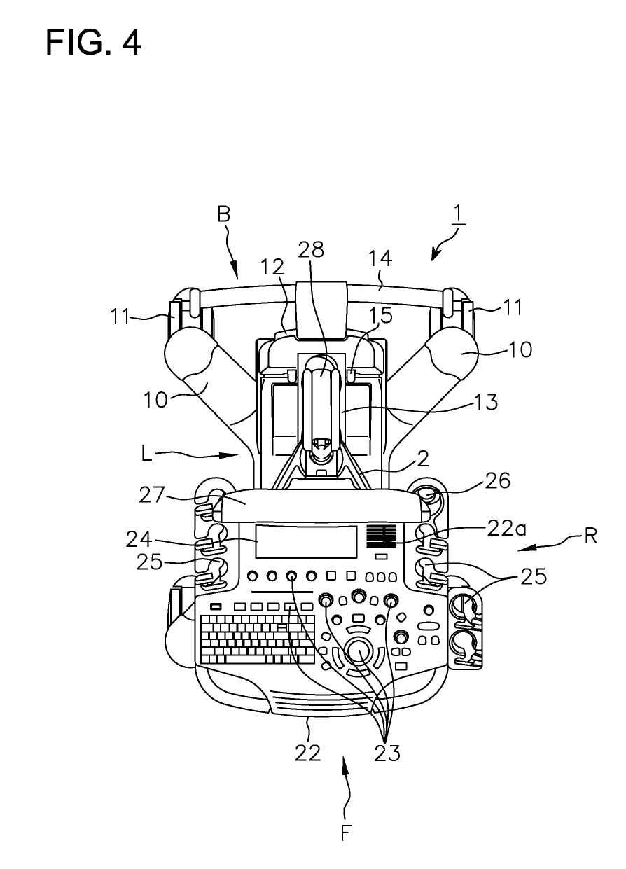

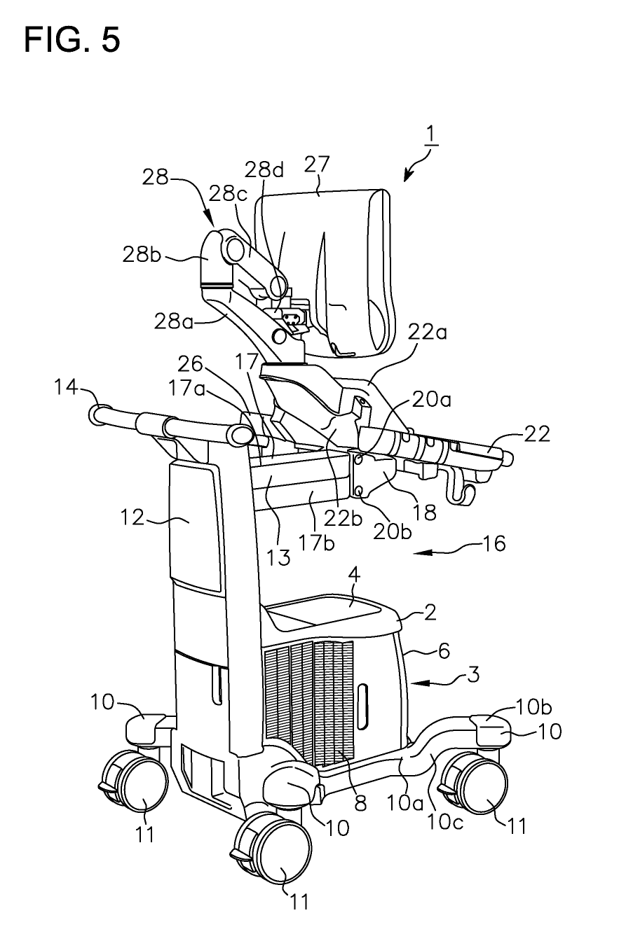

Embodiments of the present invention will be discussed based on FIG. 1 through FIG. 12 as follows. An ultrasonic diagnostic apparatus 1 shown in FIG. 1 includes a nearly cubical-shaped housing 2. The housing encloses a circuit board including an arithmetic processor that processes the echo signals acquired at an ultrasonic probe (not shown) by transmitting the ultrasonic waves to create an ultrasonic image data.

The housing 2 has four surfaces 3 (besides an upper surface 4 and a lower surface 5). An operator stands and operates an operation panel 22, in front of a front surface 6. Thus, the number 7 indicates a right side surface and the number 8 indicates a left side surface, which are of the four side surfaces 3 of the housing 2.

In the following description, in the ultrasonic diagnostic apparatus 1, the front surface 6 is referred to as a front side F of the ultrasonic diagnostic apparatus 1, the right side surface 7 is a right side R, and the left side surface 8 is a left side L of the ultrasonic diagnostic apparatus 1. Also, an opposing lateral surface of the front side F is referred to a back side B of the ultrasonic diagnostic apparatus 1.

A plurality of connectors 9 (five connectors in this example) is provided for connecting the ultrasonic prove on the right side surface 7 of the housing 2. Because the connectors 9 are provided on the right side surface 7, a width (in a horizontal direction) of the housing can be suppressed, compared to the case that the connectors 9 are provided on the front surface 6. Note that the connectors 9 can be provided on the left side surface 8.

Four wheel attachment members 10 are provided so as to protrude laterally from the housing 2 on the lower surface of the housing 2. In this example, two wheel attachment members 10 are provide each on the right side surface 7 and the left side surface 8. One wheel 11 is attached to respective wheel attachment members 10 for supporting the ultrasonic diagnostic apparatus 1 at the installation position.

The term “protruding laterally from the housing 2” refers to in the outward surrounding direction of the ultrasonic diagnostic apparatus 1. Thus, the protruding directions of the wheel attachment members 10 can be any direction with 360-degree of surrounding of the ultrasonic diagnostic apparatus 1. In this example, the wheel attachment members 10 protrude in oblique directions toward the front side F and the back side B from the right side surface 7 and the left side surface 8 in a planar view.

The wheel attachment member 10 has a sloping portion 10c that obliquely inclines upward from a base end portion 10a to a distal end portion 10b, and the height of the distal end portion 10b from the installation position of the ultrasonic diagnostic apparatus 1 is higher than the base end portion 10a. Also, the height of the distal end portion 10b from the installation position is higher than the lower surface 5 of the housing 2.

The wheel 11 is attached to an under surface 10b1 of the distal end portion 10b. Thus, the height of the under surface 10b1 to which the wheel 11 is attached is higher than the lower surface 5 of the installation position. This makes the lower surface 5 of the housing 2 closer to the installation position so that the height of the housing 2 from the installation position can be suppressed.

A board-shaped arm attachment member 12 is provided on the back side B of the ultrasonic diagnostic apparatus 1. The arm attachment member 12 is formed integral with the housing 2 and extends in upper direction from the upper surface 4 of the housing 2. An arm 13 is attached to a surface of the arm attachment member 12 facing the housing 2 and a gripper 14 is attached to the opposite surface of the arm attachment member 12. The operator can roll the ultrasonic diagnostic apparatus 1 by holding the gripper 14 and pushing the ultrasonic diagnostic apparatus 1.

The arm 13 is attached to an attachment 15 provided on the arm attachment member 12, and is rotatable upward and downward in a vertical direction. In detail, the arm 13 is attached so as to extend toward the front side F from the attachment 15. In here, the front side F is a front surface side of the ultrasonic diagnostic apparatus 1 and the back side B is a back surface side of the ultrasonic diagnostic apparatus 1. Expressed in another way, the arm 13 extends in the front-back direction of the ultrasonic diagnostic apparatus 1. The length of arm 13 is almost the same length of the length (the length in front-back direction) of the housing 2.

The attachment 15 is provided at a higher position than the housing 2. Thus, the arm 13 is positioned above the housing 2. Regarding the height of the attachment 15, it is provided at a certain height from the upper surface 4 of the housing 2. The certain height is designed for establishing a space 16 above the housing 2 that the arm 13 can rotate downward from the horizontal position X where the arm 13 is positioned horizontally.

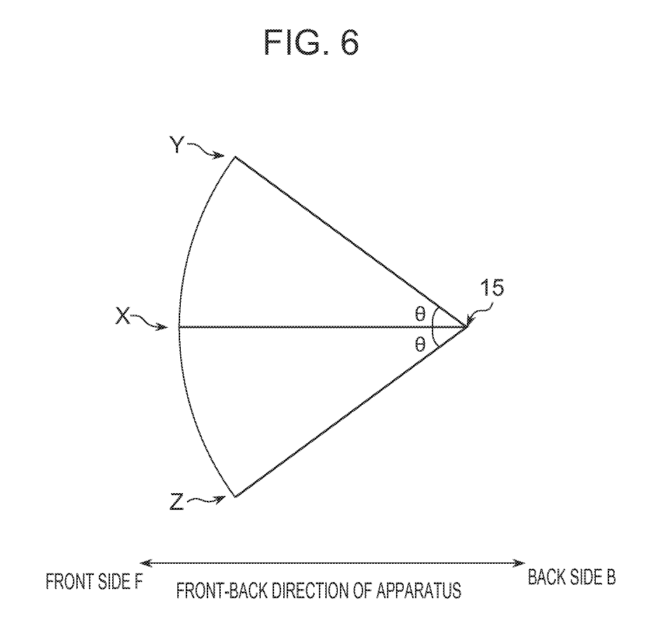

The rotation angle of the arm 13 is explained hereinafter. The arm 13 rotates within the same angle up to θ° upwardly and downwardly from the horizontal position X where the arm 13 positions horizontally as shown in FIG. 6. When the arm 13 rotates upwardly with angle of θ°, which is the highest position Y, from the horizontal position X, and when the arm 13 rotates downwardly with angle of θ°, which is the lowest position Z, from the horizontal position X. As just described, the arm 13 rotates upwardly and downwardly with the same angle of θ°. So the position in a front-back direction of the operation panel 22 attached to the end of the arm 13 at the highest position Y is the same as the position in the front-back direction of the operation panel 22 at the lowest position Z. With respect to the position of operation panel 22 in the front-back direction, the position of the operation panel 22 at the horizontal position X is farther from the attachment 15 than those positions at the highest position Y and the lowest position Z.

If the arm 13 is only rotates upwardly with 2θ°, the distance between the position of the panel 22 at the horizontal position X and at the highest position Y is longer. In the case that the arm 13 is only rotates downwardly with 2θ° is the same. When the arm 13 is rotates upwardly or downwardly respectively with each θ°, the distance between the position of the panel 22 at the horizontal position X and at the highest position Y or the lowest position Z is shorter.

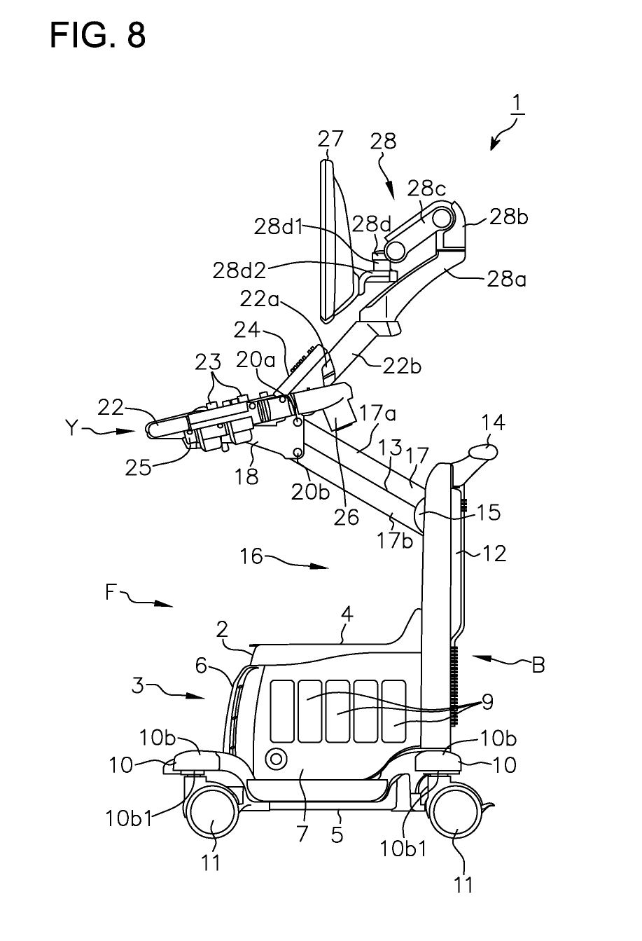

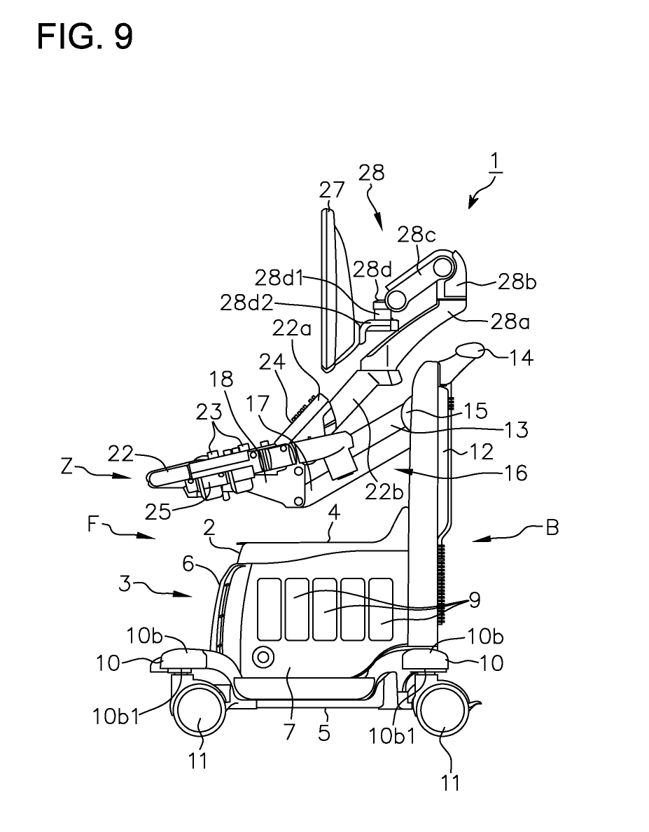

Note that in FIG. 1 through FIG. 5, the arm 13 is in the horizontal position X. In FIG. 8 described later, the arm 13 rotated to the highest position Y, and in FIG. 9 also described later, the arm 13 rotated to the lowest position Z.

An operation panel 22 is attached to the distal end of arm 13, as described below. The operation panel 22 is desirably located at a position within a range of 120 to 130 cm from the installation surface when the arm 13 is rotated to the highest position Y. Also, the operation panel 22 is desirably located at a position about 60 cm from the installation surface when it is rotated to the lowest position Z. The horizontal position X is located midway between the highest position Y and the lowest position Z.

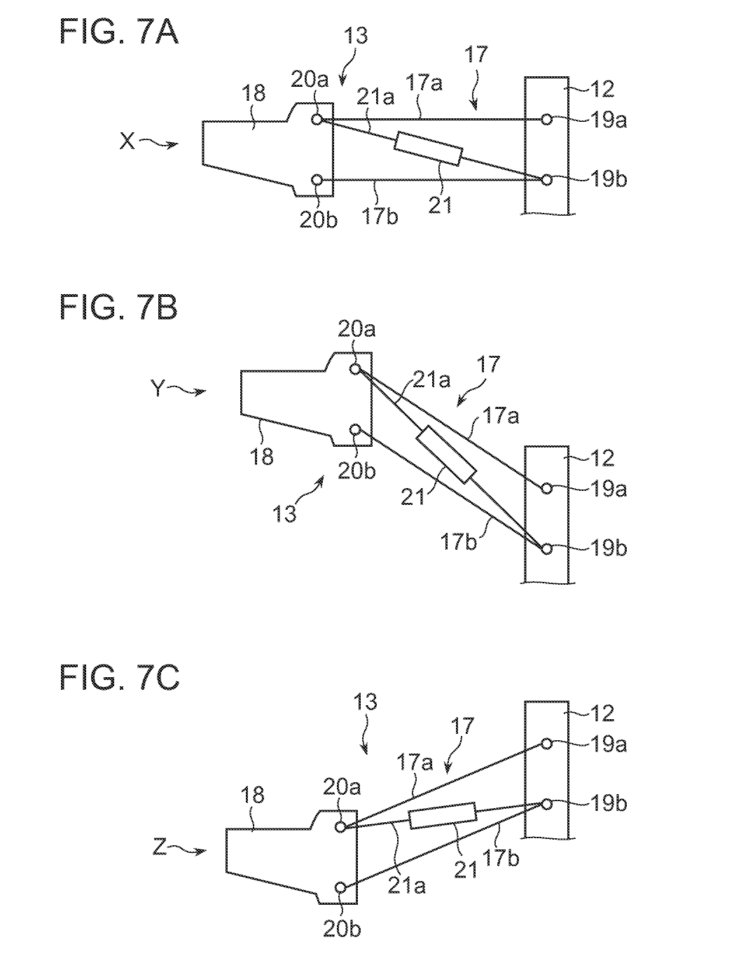

The arm 13 has a first member 17 on the base side and a second member 18 on the distal end side. The first member 17 has an upper member 17a and a lower member 17b. For more detail, as shown in FIGS. 7A, 7B, and 7C, the upper member 17a and the lower member 17b includes a parallel linking feature that respective distal ends of the upper member 17a and the lower member 17b are pivotally supported by a supporting member 19a, 19b of the arm attachment member 12 and a supporting member 20a, 20b of the second member 18. Then, the supporting member 20a of the upper member 17a on the second member 18 side and the supporting member 19b of the lower member 17b on the arm attachment member 12 side are connected by a gas spring 21. Note that FIGS. 7A-7C are schematic views for explanation.

The position of the arm 13 can be fixed by the gas spring 21. Particularly, the gas spring 21 has a lock function (not shown) to lock sliding of a shaft 21a. The gas spring 21 presses the supporting member 20a and 19b where the movement of the shaft 21 is locked, hence the position of the arm 13 is fixed.

The shaft 21a becomes slidable when the lock function is released by pressing a button on the operation panel 22. As a result, as shown in FIGS. 7A, 7B, and 7C, the arm 13 can be rotated upwardly and downwardly. Then, pushing of the button is released, the shaft 21a is locked and the position of the arm 13 is fixed.

The operation panel 22 is provided at the distal end of the second member 18. Therefore, the operation panel 22 at such position is located on the front side F of the ultrasonic diagnostic apparatus 1.

The second member 18 is provided on the lower surface of the operation panel 22. Further, the operation panel 22 is provided rotatable horizontally to the second member 18.

An operation tool 23, such as a keyboard, operation buttons, a trackball, is provided on the operation panel 22. The operation tool 23 is provided on the upper surface of the operation panel 22. Also a sub-display 24 of a touch panel is provided on the operation panel 22. Buttons for operation are displayed on the sub-display 24.

Multiple probe holders 25 for holding the ultrasonic prove are provided on both right and left sides of the operation panel 22. Also, a gel holder 26 for holding a gel container is provided on the right side of the operation panel 22.

A standing portion 22a, which stands upward, is provided at a part of the operation panel 22. The standing portion 22a is formed above the arm 13. The lower surface of the standing portion 22a has a sloping surface 22b and the sloping surface 22b is formed for not interfering with the arm 13 and the operation panel 22 when the arm 13 is rotated downwardly.

A display 27 for displaying ultrasonic images is attached to the operation panel 22 via a display attaching arm 28. The display attaching arm 28 is attached to the upper side of the operation panel 22, and has a first attaching arm 28a, a second attaching arm 28b, a third attaching arm 28c, and a display attaching member 28d. The first attaching arm 28a is attached to the standing portion 22a of the operation panel 22 rotatable in a horizontal direction. Also the first attaching arm 28a extends upward from the operation panel 22 forming an arch shape. As a result, even when the arm 13 rotates to the lowest position Z, the arm attachment member 12, the arm 13 and the first attaching arm 28a do not interfere with each other. (See FIG. 9).

The second attaching arm 28b is attached to the first attaching arm 28a and rotatable horizontally. The second attaching arm 28b is vertically-elongated from an attaching surface of the first attaching arm 28a. The third attaching arm 28c is attached to the distal end of the second attaching arm 28b. Further, the display attachment member 28d is attached at the distal end of the third attachment arm 28c.

The third attaching arm 28c rotates upwardly and downwardly from the second attaching arm 28b. The display attachment member 28d also rotates upwardly and downwardly from the third attaching arm 28c.

The display attachment member 28d has a first attachment portion 28d1 and a second attachment portion 28d2. The first attachment portion 28d1 is attached to the third attaching arm 28c and is rotatable upward and downward from the third attaching arm 28c. Further, the display 27 is attached to the second attachment portion 28d2. The second attachment portion 28d2 is rotatable horizontally from the first attachment portion 28d1, hence a direction of the display 27 can be turned to desired angles.

Operation of the ultrasonic diagnostic apparatus 1 is explained as follows. The ultrasonic diagnostic apparatus 1 has functions changing the heights of operation panel 22 and the display 27 with the movement of the arm 13. The operator setups the height of the operation panel 22 and the display 27 depending on a posture when ultrasonic scanning is conducted.

Regarding the ultrasonic diagnostic apparatus 1, the positions of the operation panel 22 and the display 27 can be set to a position corresponding to situations that the operator is in a squatting position, in a standing or sitting position when the operator operates the operation panel 22 or conducts the ultrasonic scanning Particularly, when the operator is in a standing position, the arms 13 is rotated up to the highest position Y as shown in FIG. 8 for positioning the operation panel 22 and the display 27 at the highest position. When the operator is in a sitting position, the arm 13 is positioned at the horizontal position X as shown in FIG. 1 through FIG. 5. Further, when the operator is in a squatting position, the arm 13 is rotated down to the lowest position Z as shown in FIG. 9 for positioning the operation panel 22 and the display 27 at the lowest position.

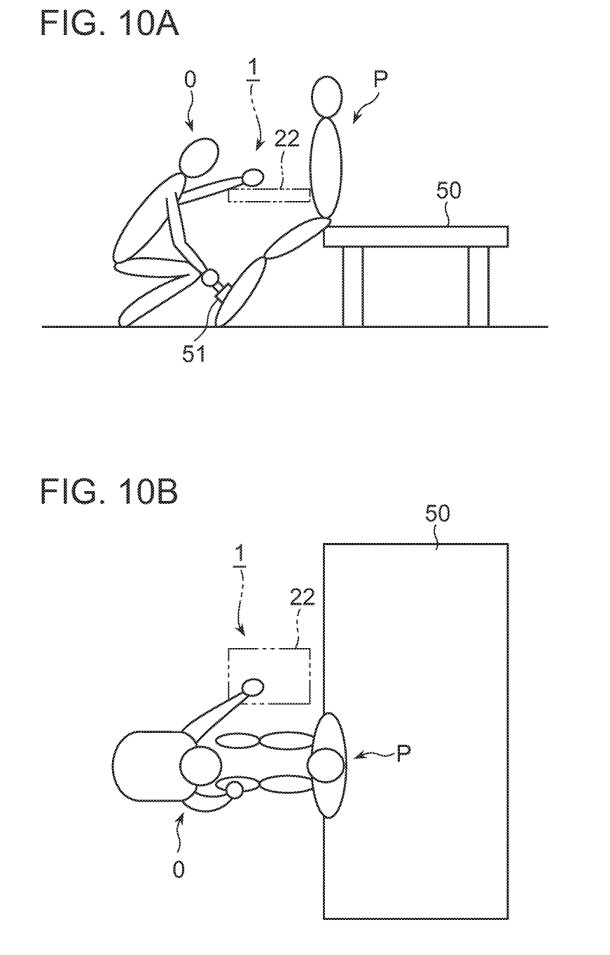

An example of usage situation of the ultrasonic diagnostic apparatus 1 when the arm 13 is rotated down to the lowest position Z is explained with FIGS. 10A and 10B. In FIGS. 10A and 10B, a patient P is sitting on a bed 50 with his/her legs on the floor. When scanning is conducted for ankles of the patient P by the ultrasonic probe 51 (shown in only FIG. 10A), the operator O faces the patient P in a squatting position to scan. The ultrasonic diagnostic apparatus 1 is placed at left side of the operator O who is facing the patient P (in FIGS. 10A and 10B, only the operation panel 22 is illustrated with a virtual line). However, the posture of the operator O and the position or the direction of the ultrasonic diagnostic apparatus 1 are as one example, and it is not limited by this illustration.

By positioning the arm 13 at the lowest position Z, the height of the operation panel 22 moves to about 65 cm. Therefore, if the operator rotates the arm 13 at the lowest position Z in a squatting position, the operator can operate the operation panel 27 in a proper posture. Also the display 27 moves to the lowest position where the operator sees it easily.

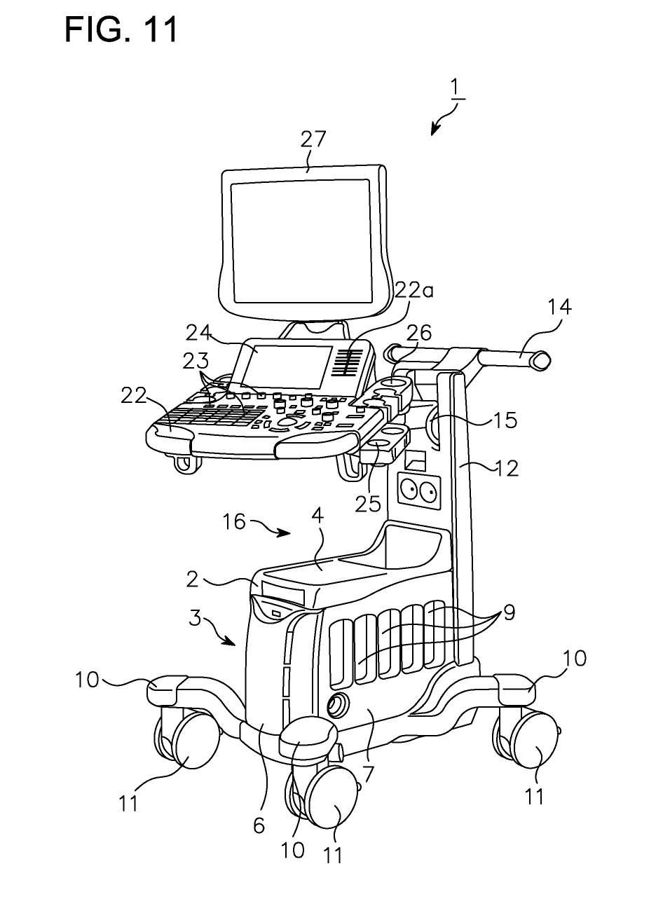

The operator can rotate the operation panel 22 horizontally such that the operation panel 22 faces a suitable operating position for the operator such as shown in FIG. 11.

According to the ultrasonic diagnostic apparatus 1 of the present embodiment, the arm 13 is attached to the attachment 15 of the arm attachment member 12 provided on the back side B. The attachment 15 is provided at a higher position than the housing 2, thus the arm 13 is located above the housing 2. Therefore, the arm 13 rotates upwardly and downwardly above the housing 2 so that the length (a length in a direction of front and back) of the ultrasonic diagnostic apparatus 1 can be suppressed. Further, the arm 13 also rotates upwardly and downwardly from the horizontal position X so that the rotating range of the operation panel 22 in the front-back direction by the arm 13 rotating can be small. Accordingly, the length in a direction of front and back of the ultrasonic diagnostic apparatus 1 is suppressed and it can be installed into a small space.

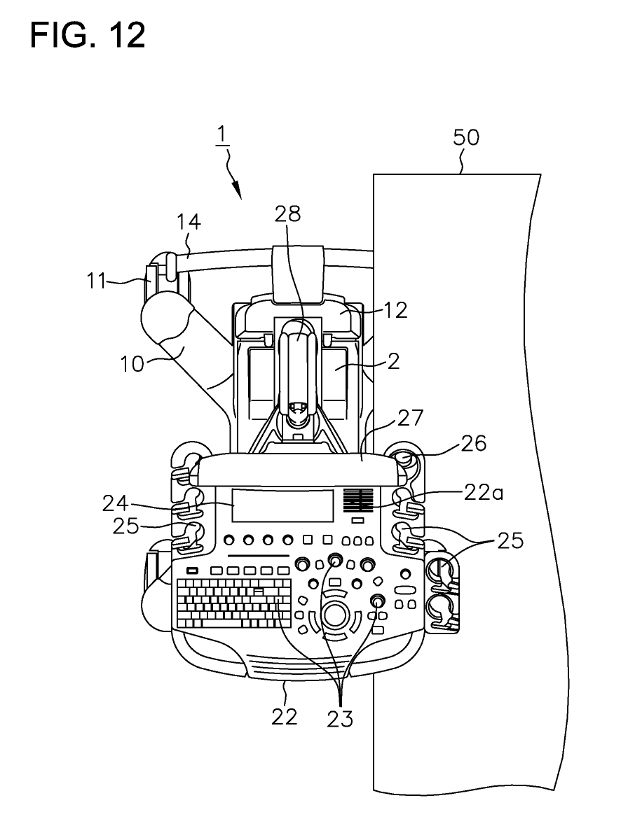

A plurality of connectors 9 are provided on the right side of the housing 2 of the ultrasonic diagnostic apparatus 1. Thus the width of the housing 2 can be suppressed. Further, the wheel attachment members 10 are provided so as to protrude laterally, thus stability of the ultrasonic diagnostic apparatus 1 can be ensured even if the wide of the housing 2 is narrowed. Also, as shown in FIG. 12, the ultrasonic diagnostic apparatus 1 is positioned such that the wheel attachment members 10 go under the bed 50, the ultrasonic diagnostic apparatus 1 can be closer to the bed side. As a result, operability of the operation panel 22 and the visibility of the display 27 can be improved for the operator. Also, even a size of bed side is narrow, it can be installed.

While preferred embodiments and examples of the current invention have been described above, it will be understood by those skilled in the art that additional modifications and changes can be made to the embodiments and examples without departing from the spirit of the invention. For example, in the above-mentioned embodiment, the arm 13 rotates the same angle of θ° to upward and downward from the horizontal position X, but the rotating angle to upward and to downward can be different.

1. An ultrasonic diagnostic apparatus comprising:
a housing;
an arm attached to an attachment provided above the housing, the arm configured to rotate upward and downward, the arm extending in a direction of front and back of the attachment; and
an operation panel provided at a distal end of the arm, wherein the attachment is oriented at a certain position from an upper surface of the housing such that the arm is rotatable upward and downward from a horizontal position.

2. The ultrasonic diagnostic apparatus of claim 1, wherein the attachment is provided on an arm attachment member extending upward from the upper surface of the housing.

3. The ultrasonic diagnostic apparatus of claim 2, wherein the arm attachment member is provided on a back side of the ultrasonic diagnostic apparatus and the arm is formed to extend from the back side to a front side of the ultrasonic diagnostic apparatus.

4. The ultrasonic diagnostic apparatus according to claim 1, wherein a height of the attachment from the upper surface of the housing defines a space for ensuring the rotating movement of the arm is created above the housing.

5. The ultrasonic diagnostic apparatus according to claim 1, wherein a first rotating angle upward from the horizontal position and a second rotating angle downward from the horizontal position of the arm are the same.

6. The ultrasonic diagnostic apparatus according to claim 1, wherein a first rotating angle upward from the horizontal position and a second rotating angle downward from the horizontal position of the arm are different.

7. The ultrasonic diagnostic apparatus according to claim 1, further comprising a plurality of wheels configured to support the ultrasonic diagnostic apparatus at an installation surface, each of the plurality of wheels attached to a respective wheel attachment member extending laterally from the housing.

8. The ultrasonic diagnostic apparatus of claim 7, wherein a height of an attachment surface of each wheel on the respective wheel attachment member is higher than a lower surface of the housing.

9. The ultrasonic diagnostic apparatus according to claim 1, wherein the housing is substantially cube-shaped and has four side surfaces, the housing comprising at least one connector for an ultrasonic probe formed either on a right side surface or a left side surface adjacent to a front side surface, where an operator operates the operation panel.

10. The ultrasonic diagnostic apparatus according to claim 1, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

11. The ultrasonic diagnostic apparatus according to claim 1, further comprising a parallel linking feature configured to enable the arm to rotate.

12. The ultrasonic diagnostic apparatus according to claim 1, wherein the housing encloses a circuit board comprising an arithmetic processor configured to process echo signals acquired by transmitting ultrasonic waves.

13. The ultrasonic diagnostic apparatus according to claim 2, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

14. The ultrasonic diagnostic apparatus according to claim 3, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

15. The ultrasonic diagnostic apparatus according to claim 4, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

16. The ultrasonic diagnostic apparatus according to claim 5, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

17. The ultrasonic diagnostic apparatus according to claim 6, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

18. The ultrasonic diagnostic apparatus according to claim 7, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

19. The ultrasonic diagnostic apparatus according to claim 8, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.

20. The ultrasonic diagnostic apparatus according to claim 9, further comprising a display attached to the operation panel via a display attaching arm, wherein the display attaching arm is attached to an upper surface side of the operation panel and is formed upward so as not to interfere with the arm even when the arm rotates to a lowest position and the display attaching arm extends above the operation panel.
